(19) United States
(12) Patent Application Publication
(10) Pub. No.: US 2019/0170771 A1
(43) Pub. Date: Jun. 6, 2019

(54) ALPHA-SYNUCLEIN IN PERIPHERAL BLOOD MONONUCLEAR CELLS AS BIOMARKER FOR SYNUCLEINOPATHY

(71) Applicant: Lysosomal and Rare Disorders Research and Treatment Center, LLC., Fairfax, VA (US)

(72) Inventors: Renuka Pudi LIMGALA, Fairfax, VA (US); Ozlem GOKER-ALPAN, Fairfax, VA (US)

(21) Appl. No.: 16/321,242

(22) PCT Filed: Jul. 28, 2017
(86) PCT No.: PCT/US2017/044430

§ 371 Date: Jan. 28, 2019

Related U.S. Application Data
(60) Provisional application No. 62/368,129, filed on Jul. 28, 2016.

(51) Int. Cl. G01N 33/68; G01N 15/14

(52) U.S. Cl. CPC G01N 33/6896; G01N 2015/1488; G01N 2496/05; G01N 15/14

(57) ABSTRACT

Disclosed is a rapid, non-invasive and highly specific and sensitive diagnostic assay for the identification of individuals with synucleinopathy and for measuring synucleinopathy progression or status. Test kits for diagnosis of an individual suspected of having synucleinopathy are also disclosed.

### RELATED APPLICATION

This Application claims the benefit of priority to U.S. Provisional Application 62/368,129 filed Jul. 28, 2016 in the U.S. Patent and Trademark Office, the disclosure of which is incorporated by reference herein in its entirety.

### BACKGROUND

GBA (OMIM 606463) gene codes for Glucosidase, beta, acid or beta-glucocerebrosidase, a lysosomal enzyme. In this disclosure, the product of the GBA gene is referred to herein as glucocerebrosidase. Disease causing mutations in both alleles of GBA gene cause Gaucher disease (GD) while mutations in one allele lead to Gaucher carrier status. It has been shown recently that patients with GD, even carriers with one mutated GBA gene are at a higher risk for developing synucleinopathy (e.g., Parkinson disease (PD)), and at an earlier age (Goker-Alpan et. al., 2004), and the GBA mutations comprise the primary genetic risk factor in the development of PD and other forms of parkinsonism (i.e., types of synucleinopathy Sidransky et. al., 2009). However, there are no biomarkers to determine the diagnosis, especially in the early and minimally symptomatic or asymptomatic stage or follow the progression of synucleinopathy such as PD in subjects with a mutation in the GBA gene. In other cellular and animal models, glucocerebrosidase alterations were shown to impact the metabolism of other proteins implicated in PD pathology (Mazzulli et al., 2011). Alpha-Synuclein and Parkin, encoded by SNCA and PARK2 respectively, are implicated in rare genetic forms of parkinsonism when they are present in brain tissue. Alpha-Synuclein aggregates are seen in cells of central and peripheral nervous system and is considered to be the pathological culprit in PD (Giraldez-Perez et. al., 2013), and the mutated glucocerebrosidase has been shown to be present in Alpha-Synuclein aggregates in postmortem brain samples from individuals with GBA mutations and PD (Swan & Saunders-Pullman, 2014). Alpha-Synuclein in addition, is shown to affect the solubility of Parkin in the cells (Kawahara et. al., 2008). As an attempt to assess whether GBA alterations would also impact Alpha-Synuclein and Parkin metabolism in humans in easily accessible cell types outside the brain, we investigated the expression at protein level in the peripheral blood mononuclear cells (PBMCs) using Flow cytomtery.

Synucleinopathies (also called α-Synucleinopathies) are neurodegenerative diseases characterized by the abnormal accumulation of aggregates in brain including in neurons nerve fibers or glial cells. Synucleinopathy encompasses a group of neurodegenerative disorders such as Parkinson disease, Dementia with Lewy Bodies and Multiple System Atrophy. There is now evidence that the phenotype associated with glucocerebrosidase (GBA) mutations include the entire spectrum of synucleinopathies (Eblan et al., 2005).The prototype synucleinopathy, also one of the most common neurodegenrative disorders is Parkinson Disase (PD). In PD, the degeneration of the dopaminergic neurons accompanied by the formation of alpha-synuclein containing Lewy bodies results in a marked loss of dopamine. Early non-motor signs of PD are suggested to include olfactory dysfunction, mood and behavioral disturbances, subtle neurocognitive dysfunction and visual motor control abnormalities (Wolters et al. 2000). However these signs are neither specific nor sensitive. In addition, the currently accepted clinical criteria for the diagnosis of PD generally have poor specificity for differentiating other parkinsonian syndromes. The duration of the preclinical phase between the onset of pathological changes and the loss of significant striatal dopamine resulting in parkinsonian manifestations may be variable. The dopamine concentrations must be significantly reduced, usually by more than 50%, for classic clinical signs to develop (Guttman et al., 1997). Although several clinical studies have suggested that the prodromal period might have been as long as several decades, recent postmortem and imaging data (PET) indicate that this latency period is quite variable and might be even shorter than five years (Morrish et. Al., 1998). There are several implications of preclinical phase, especially in developing protective therapies. There are no current biomarkers in Parkinson disease and other related synucleinopathies that indicate and correlate with disease onset, activity and progression. Biomarkers are used to diagnose, and/or monitor the disease progression, and therapeutic response clinically. Biomarkers are also essential in drug development for both curative or preventative therapies.

To date, there is no minimally invasive, accurate test for the diagnosis of synucleinopathy including, for example, Parkinson disease; and especially for synucleinopathy in patients that carry a mutation in one or two alleles of the GBA gene. Furthermore, there is no accurate diagnostic test which does not involve the use of brain cells or central nervous system cells and in particular, which only uses cells from the peripheral blood such as peripheral blood mononuclear cells.

### BRIEF DESCRIPTION

The present invention relates to a method for diagnosing synucleinopathy (the singular form of synucleinopathies), for diagnosing the severity or speed of progression of synucleinopathy, kits for performing the methods, and a method for monitoring the effectiveness of treatment of synucleinopathy.

Synucleinopathy in this disclosure refers to one or more of neurodegenerative diseases whose main pathogenesis is a result of abnormal accumulation of alpha-synuclein in brain tissue. Synucleinopathy include but not limited to one or more of PD, Parkinsonism, dementia with Lewy bodies and multiple system atrophy.

One embodiment of the invention relates to a method to diagnose synucleinopathy in a first subject comprising two steps. The first step involves detecting a lymphocyte expression level of Alpha-Synuclein or a monocyte expression level of Alpha-Synuclein in a first subject. The second step involves comparing the lymphocyte expression level of Alpha-Synuclein or the monocyte expression level of Alpha-Synuclein with a second subject who does not have synucleinopathy wherein an increase in the expression level of Alpha-Synuclein in the first subject relative to the second subject indicates that the subject has synucleinopathy. Synucleinopathy can be at least one selected from the group consisting of Parkinson Disease, Parkinsonism, dementia with Lewy bodies; and multiple system atrophy

The subject may be any mammal preferably a human. Other mammals include those mammals that are used as disease models for synucleinopathy such as Parkinson Disease.

The detecting step may involve collecting a blood sample such as whole blood or peripheral blood from a subject. A preferred amount of blood would be from 1 ml to about 5 ml such as for example, around 2 ml, 3 ml, or 4 ml. In a preferred embodiment, the blood sample may be enriched for mononuclear cells. For example, the sample may be peripheral blood and it may be enriched for peripheral blood mononuclear cells. Enrichment may be performed using commercially available methods. In the method, a similar sample may be obtained from the first and/or the second subject.

In the method, a ratio of the expression level of Alpha-Synuclein in the first subject to the expression level of Alpha-Synuclein in the second subject (the control non-synucleinopathy subject) can be calculated. This calculation may be based on Alpha-Synuclein expression levels in lymphocytes, or in monocytes, or both cell types. We have found surprisingly that Alpha-Synuclein expression in in lymphocytes, or in monocytes, preferably in the peripheral blood mononuclear cells, are correlated with a synucleinopathy disease state. Therefore, in particular, a ratio of at least 1.5:1 or at least 2:1 in lymphocytes or in monocytes confirms a synucleinopathy (especially a Parkinson Disease) diagnosis. In a preferred embodiment, a ratio of at least 1.2:1, at least 1.3:1, at least 1.4:1, at least 1.5:1, at least 1.7:1, at least 2:1 or at least 3:1 in lymphocytes and in monocytes confirms a synucleinopathy (especially a Parkinson Disease) diagnosis. Of these values, at least 1.5 and at least 2 is preferred. That is, a synucleinopathy lymphocyte or monocyte expresses Alpha-Synuclein at a level of at least 110%, 120%, 130%, 140%, 150%, 170%, 200% or 300% more than that of a corresponding non-synucleinopathy cell lymphocyte or monocyte.

In an embodiment, the step of detecting is performed by flow cytometry, for example, using a flow cytometer. The data can be analyzed using a FSC vs SSC scatter plot followed by scatter plot or histogram to quantitate fluorescence, and the expression level or relative expression level quantified for diagnosis. In other embodiments, any method for detection of protein expression may be used. These methods include HPLC, mass spectrometry, immunoprecipitation, dip stick assays, automated slide scanning, sandwich assays, and the like.

In the method of claim 1, the detecting step may be performed with a commercially available anti-Alpha-Synuclein antibody. The antibody can be tagged by a detectable label. A detectable label may be, for example, a fluorescent tagged antibody, a radioactively tagged label etc. Alternatively the antibody may be tagged by the binding of a secondary antibody to it wherein the secondary antibody is tagged with a detectable label. The antibody can be a complete antibody or an engineered antibody. For example, the antibody may comprise the antigen binding parts of an antibody molecule (Fab for example) or might be a single chain antibody or a multimeric antibody.

The methods of the invention are especially accurate if it is used to diagnose subjects who have GBA gene mutations, Gaucher Disease, or Gaucher carrier status. Thus, in a preferred embodiment, the first subject, the second subject, or both subjects may have at least one mutated GBA gene, Gaucher Disease, or Gaucher carrier status. The at least one mutated GBA gene may encode a glucocerebrosidase with a reduced glucosylceramide cleavage activity.

In a preferred embodiment, the methods of the invention are practiced without the collection of any brain tissue or central nervous tissue. In another preferred embodiment, the only tissue collected from a subject for any of the method of the invention is peripheral blood. In one embodiment, the only tissue used for any of the methods of the invention is peripheral blood mononuclear cells.

Another embodiment of the invention relates to a method for monitoring the progression of synucleinopathy in a subject comprising the steps of: a) detecting a first lymphocyte Alpha-Synuclein expression level or a first monocyte Alpha-Synuclein expression level from a first sample from said subject; and b) determining the progression of synucleinopathy in the subject based on comparing said first lymphocyte Alpha-Synuclein expression level or said first monocyte Alpha-Synuclein expression level with a second expression level (i.e., a lymphocyte Alpha-Synuclein expression level or a monocyte Alpha-Synuclein expression level) from a second sample from the same subject collected earlier or from a second subject with synucleinopathy; wherein a high first expression level relative to said second expression level indicates a more rapid progression of synucleinopathy and a low expression level relative to said second expression level indicates a less rapid progression of synucleinopathy. The collection of samples and cells and the method of detection may be any method discussed in this disclosure including in particular any of the method discussed above.

In the case where expression level is compared with the expression level of a second subject with synucleopathy, the synucleopathy can be staged. That is, for example, if the staging of the second subject with synucleopathy is known, the expression level of lymphocyte Alpha-Synuclein or of monocyte Alpha-Synuclein or both may be compared to stage the disease of the subject. Also, in the case where expression level is compared with the expression level of a second expression level from a second sample from the same subject collected earlier (at an earlier time), if the staging of the synucleinopathy at an earlier time is known, the expression level can be compared to stage the current stage of synucleinopathy.

In any of the methods of this disclosure, a preferred embodiment is for Parkinson Disease, for example, in the detection, diagnosis and disease staging of Parkinson Disease. It follows that a preferred synucleinopathy is therefore Parkinson Disease.

In a preferred embodiment, the subject has Gaucher Disease or has Gaucher carrier status. In another embodiment, the subject has at least one mutated GBA gene. At least one mutated GBA gene can encode a glucocerebrosidase with a reduced glucosylceramide cleavage activity.

Another embodiment is related to a kit for the diagnosis of synucleinopathy in a subject comprising: a) a first reagent for the detection of Alpha-Synuclein expression a cell; and b) a second reagent selected from the group consisting of a positive control reagent and a negative control reagent. The positive control reagent may comprise lymphocytes or monocytes from peripheral blood of a second subject which has at least one of synucleinopathy, Gaucher Disease or Gaucher carrier status. In a preferred embodiment, the subject has synucleinopathy and one of Gaucher Disease or Gaucher carrier status. The negative control reagent comprises lymphocytes and monocytes from peripheral blood of a third subject which does not have Gaucher Disease, Gaucher carrier status or synucleinopathy.

The methods of this disclosure may be performed on any mammal and preferably a human although many mammals, especially mammals that are animal models for synucleinopathy. The terms “subject” and “patient” are used interchangeably in this disclosure.

The antibodies used in this disclosure are recited in the Examples section but they are also commercially available. For example, anti-Parkin antibodies are commercially available from many sources (e.g., Abexa Ltd; Abbiotec; Abcam; ABclonal; Acris Antibodies GmbH; AMSBIO LLC; Aviva Systems Biology; BioLegend; Bio-Rad; Biorbyt; Bioss Inc.; Bioworld Technology; Bosterbio; Cell Applications; Cell Signaling Technology; Creative Diagnostics; Elabscience Biotechnology Co., Ltd.; Enzo Life Sciences, Inc.; EpiGentek; FabGennix International, Inc.; Fitzgerald Industries International; GeneTex; GenScript USA Inc.; GenWay Biotech, Inc.; Immuno-Biological Laboratories—America; Invitrogen Antibodies; LifeSpan BioSciences; Novus Biologicals; NSJ Bioreagents; OriGene Technologies; ProSci, Inc; Proteintech Group Inc; Raybiotech, Inc.; Rockland Immunochemicals, Inc.; Santa Cruz Biotechnology, Inc.; Takara Bio; or United States Biological). Anti-Alpha-Synuclein antibodies are also commercially available from many sources (e.g., Abcam; Acris Antibodies GmbH; Agrisera; Alomone Labs, Ltd.; AMSBIO LLC; Atlas Antibodies; Aviva Systems Biology; BD Biosciences; BioLegend; Bio-Rad; Bosterbio; Cedarlane; Cell Sciences; Creative Biolabs; Creative Diagnostics; Dianova GmbH; Elabscience Biotechnology Co., Ltd.; Fitzgerald Industries International; GenWay Biotech, Inc.; Immuno-Biological Laboratories—America; LifeSpan BioSciences; MBL International; MyBioSource.com; OriGene Technologies; Raybiotech, Inc.; Takara Bio; United States Biological; Wako Chemicals USA, Inc.; or Wuhan Fine Biotech Co., Ltd.).

### BRIEF DESCRIPTION OF THE FIGURES

FIG. 1 PBMCs from four cohorts NonGD-nonPD, GD-nonPD, NonGD-PD and GD-PD are assayed for Parkin using flow cytometry as mentioned in the methodology. In grey, are the control plots or background fluorescence from no-primary antibody tubes. Black filled plots indicate intracellular Parkin expression.

FIG. 2 PBMCs from four cohorts NonGD-nonPD, GD-nonPD, NonGD-PD and GD-PD are assayed for Alpha-Synuclein using flow cytometry. In grey, are the control plots or background fluorescence from no-primary antibody tubes. Black filled plots indicate intracellular Parkin expression.

FIG. 3 The mean fluorescence intensity for the Alpha-Synuclein plots is quantified relative to background fluorescence in each subject in FIG. 2. The absolute values are indicated at the bottom of each plot.

FIG. 4 PBMCs from subject GD-PD-1 from three visits over a period of 3 years are assayed at the same time for Alpha-Synuclein and the results from lymphocytes and monocytes are shown. At visit-1, the subject had very early signs of PD symptoms which progressively worsened to Hoehn and Yahr stages 1 and 4 at visit-2 and visit-3 respectively.

FIG. 5 PBMCs from subject GD-PD-2 from three visits over a period of 2 years are assayed at the same time for Alpha-Synuclein and the results from lymphocytes and monocytes are shown. Subject GD-PD-2 has been diagnosed with Hoehn and Yahr stage 2 at all three visits.

FIG. 6: The results from FIGS. 4&5 are quantified as relative expression of Alpha-Synuclein over the background fluorescence for each visit and plotted. The absolute relative values are mentioned at the bottom of the histograms. The Hoehn and Yahr stages for each subject and visit are also labelled at the bottom of the plot.

### DETAILED DESCRIPTION OF THE INVENTION

This invention generally relates to methods, reagents and kits that are useful for the detection of synucleinopathy (e.g., Parkinson Disease). The invention can be used to identify patients that have Parkinson Disease (e.g., a diagnostic, prognostic or monitoring method) and to further understand the mechanisms of Parkinson Disease for the development of therapeutic strategies directed at preventing or treating this disease.

The present inventors have shown that Alpha-Synuclein is highly useful peripheral blood mononuclear cells biomarker to identify synucleinopathy (e.g., Parkinson Disease) especially in, but not necessarily in, Gaucher Disease or Gaucher Carrier Status patients.

More particularly, the present inventors compared the expression of two biomarkers Alpha-Synuclein and Parkin in the peripheral blood mononuclear cells of Gaucher Disease or Gaucher Carrier Status patients and show that the expression of Alpha-Synuclein shows distinct patterns which can be used to diagnose synucleinopathy (e.g., Parkinson Disease).

In one aspect of the invention, the cells used in the assay are lymphocytes and monocytes. One advantage of the present invention is that the cells, lymphocytes and monocytes, can be provided in the form of whole blood or peripheral blood or a fraction thereof from a patient and such samples can be collected with minimal invasiveness and inconvenience to the patient.

The method of the present invention is particularly useful because it can identify patients that have synucleinopathy in a rapid, in vitro assay that is minimally invasive and is capable of rapid diagnosis. The method is more sensitive and much less invasive than current methods of diagnosing synucleinopathy (e.g., Parkinson Disease). Moreover, prior to the present invention, the use of biomarkers in peripheral blood to diagnose synucleinopathy (e.g., Parkinson Disease) had not been described.

The experiments are performed as described in the Example Section of this disclosure.

### Results:

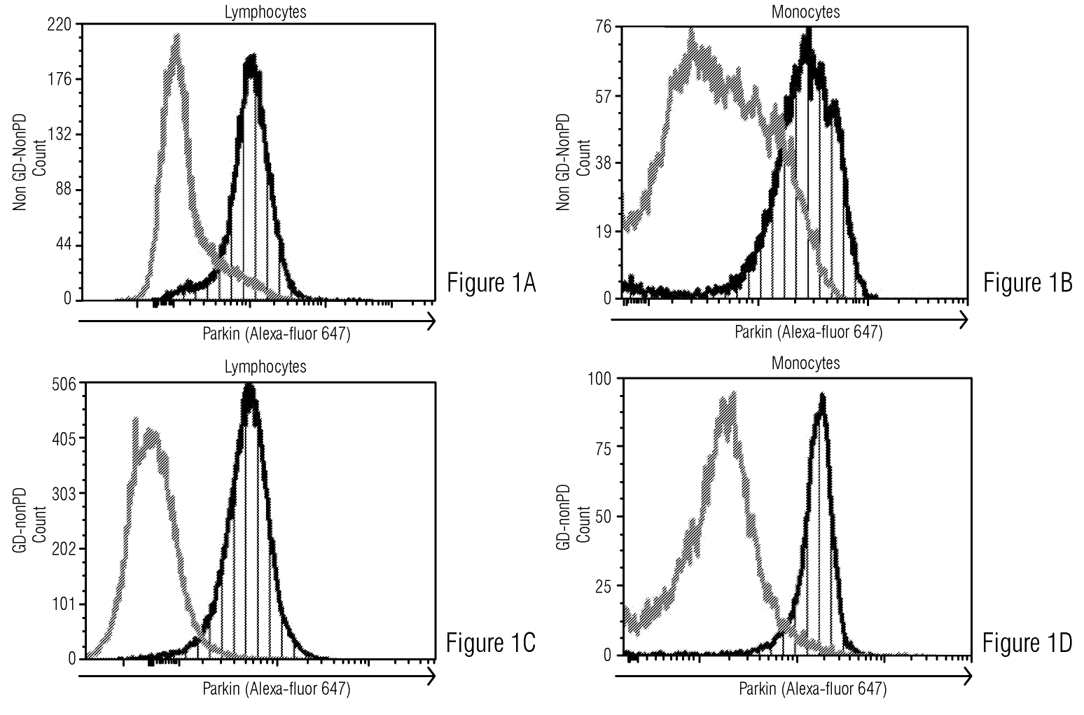

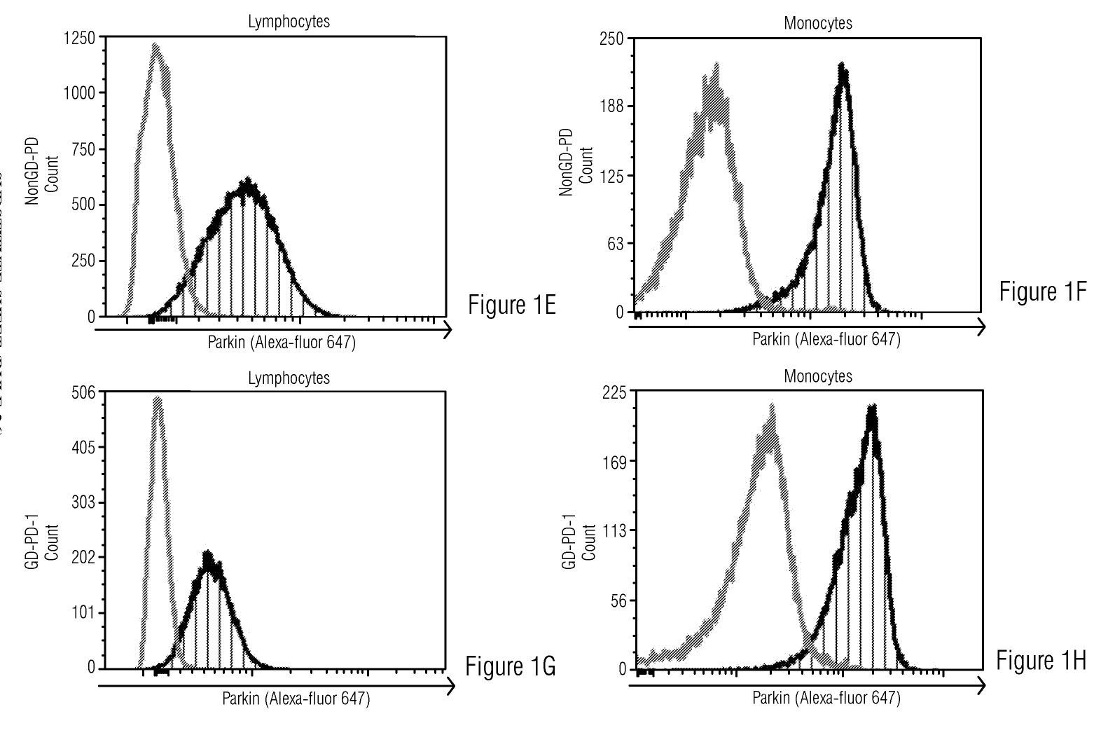

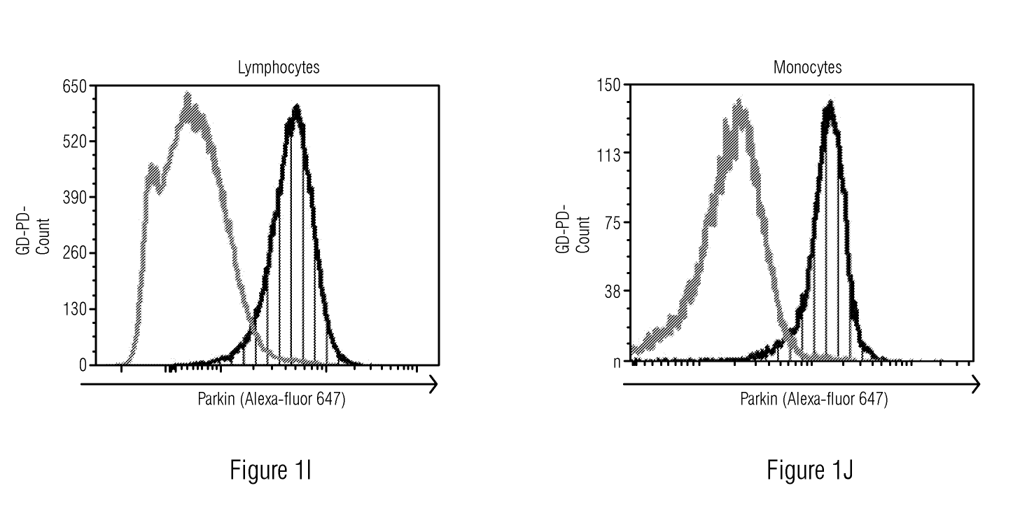

When Parkin expression was assessed, compared to nonGD-nonPD subjects, all other three cohorts showed higher expression of Parkin, especially in monocytes (FIG. 1A-J). So, even though Parkin is elevated in GD and PD subjects compared to normal controls, Parkin alone may not be sufficient to be used as a biomarker.

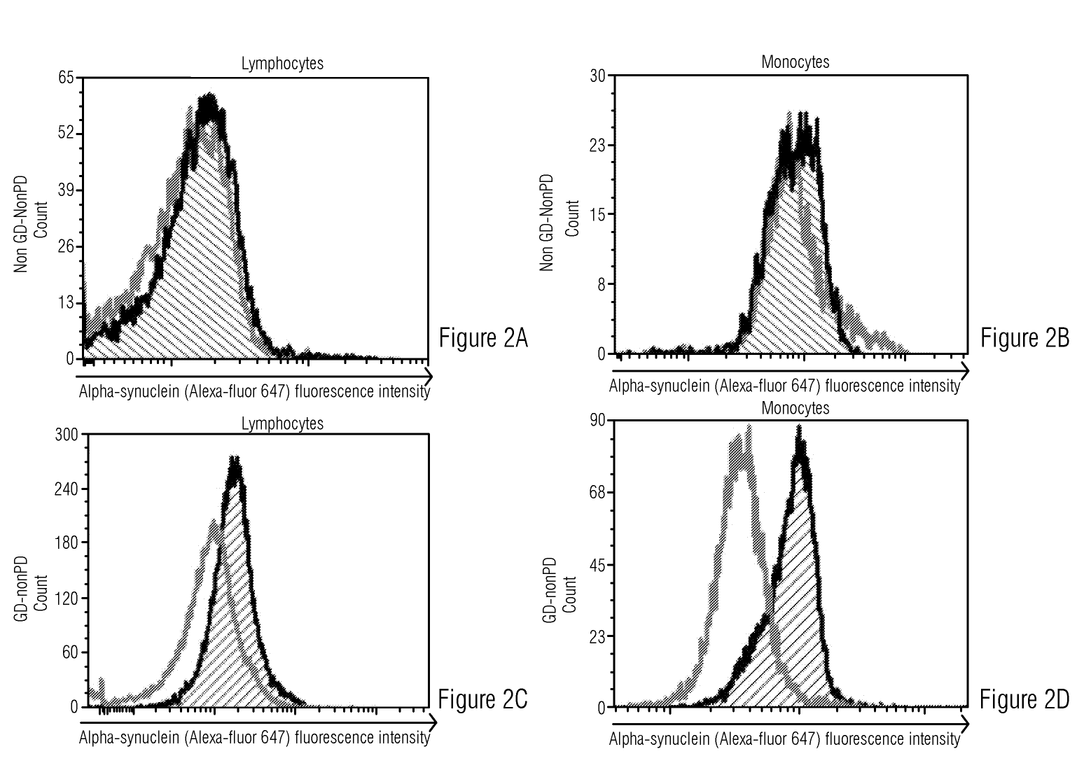

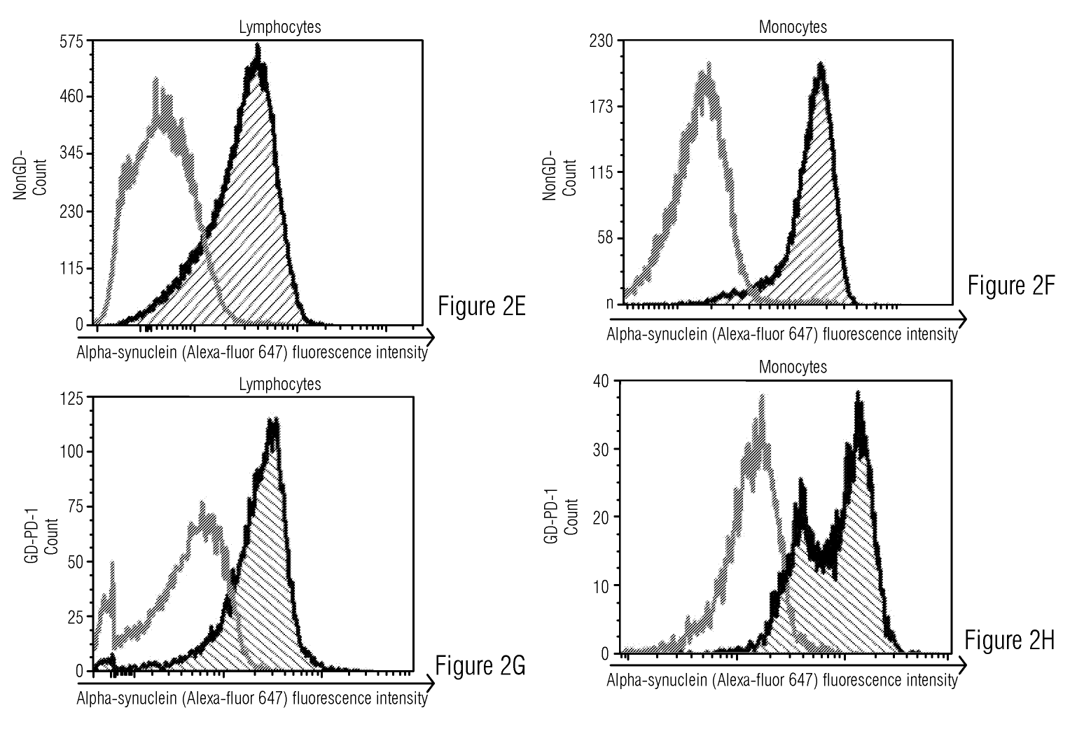

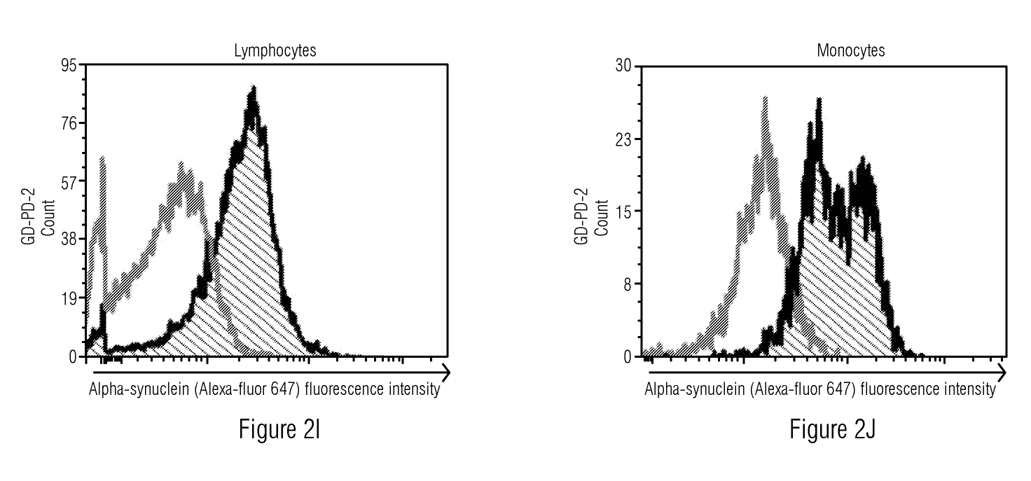

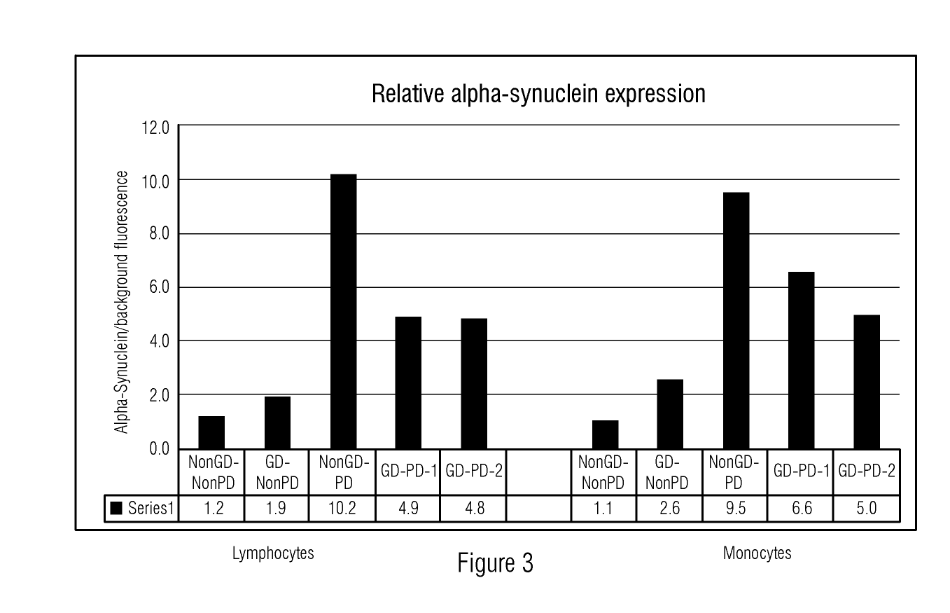

We then looked at Alpha-Synuclein expression in lymphocytes and monocytes of the samples and quantified the results as relative Alpha-Synuclein expression over the background fluorescence for each sample (FIGS. 2&3). Alpha-Synuclein expression in control subjects, i.e., nonGD-nonPD group was comparable to background fluorescence in both lymphocytes and monocytes. In GD-nonPD group, even though the alpha-synculein expression is seen, it is still minimal and overlaps significantly with the background fluorescence. In PD subjects with no known GD symptoms or mutations, there seems to be a distinct peak in Alpha-Synuclein associated fluorescence. Similar to nonGD-PD group, GBA associated PD patients also showed elevated expression of Alpha-Synuclein in lymphocytes and monocytes. In addition, in monocytes on GD-PD patients, there seem to be a distinct subgroup of monocytes with elevated expression of Alpha-Synuclein. This pattern was unique to GD-PD group and not seen in any other control groups. However, when the quantity and pattern of both Alpha-Synuclein and Parkin are studied, GBA associated PD subjects show a distinct pattern.

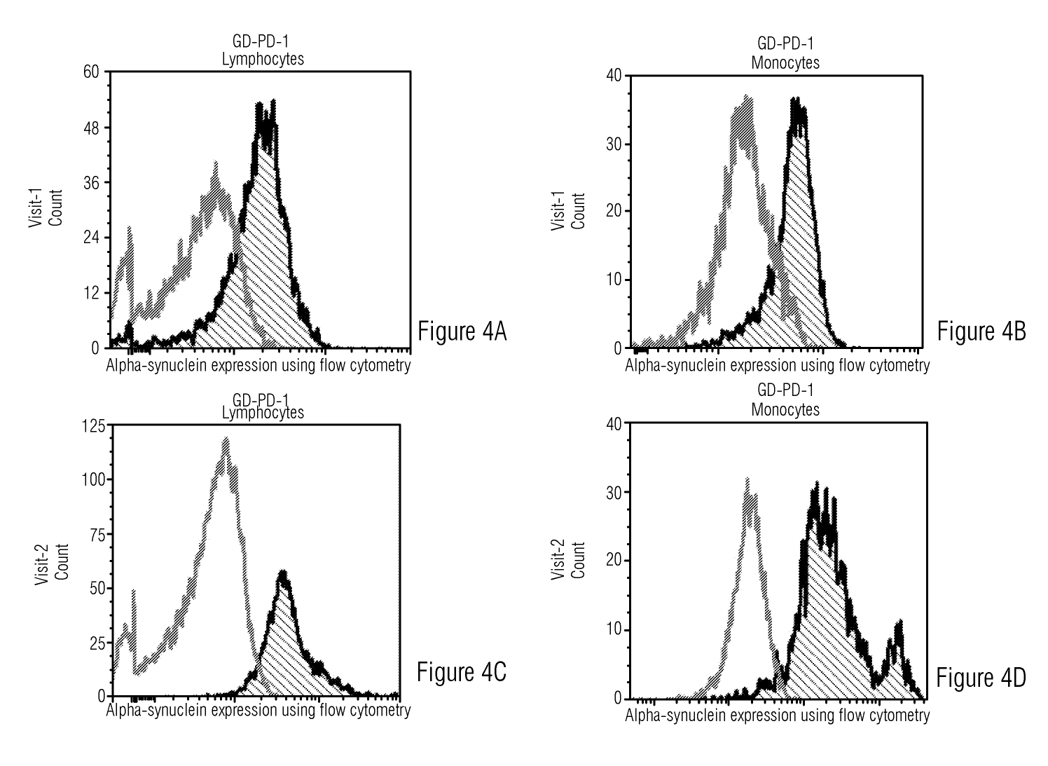

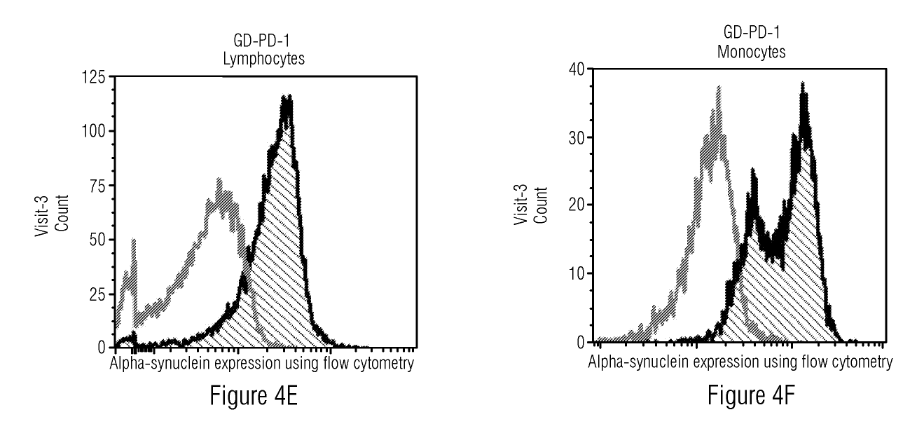

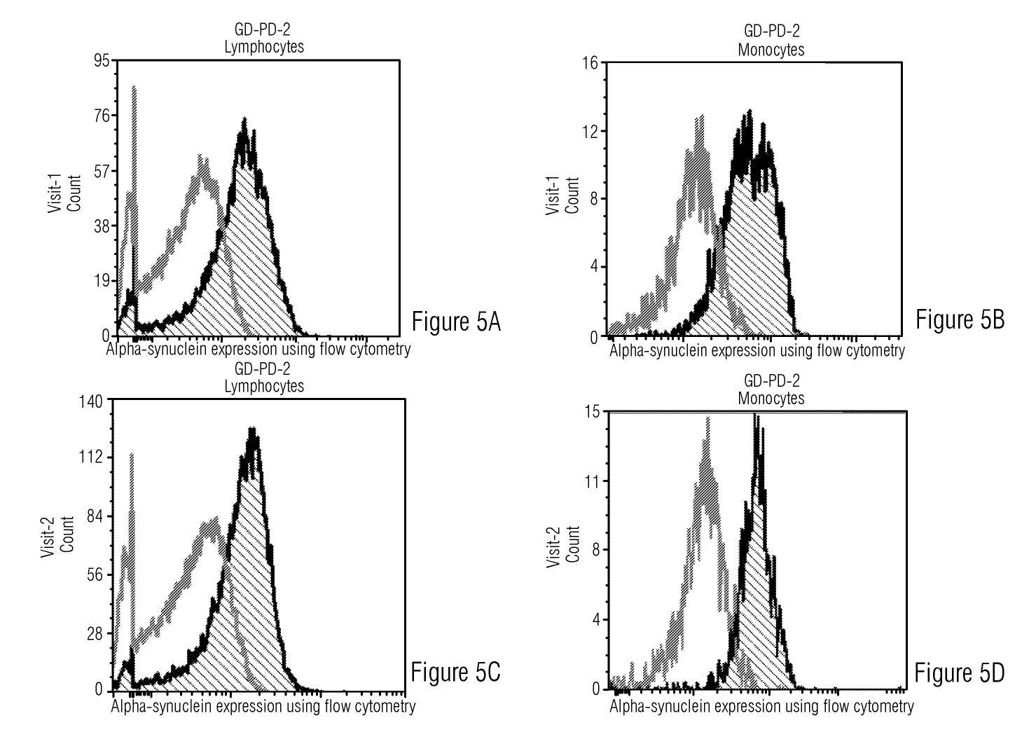

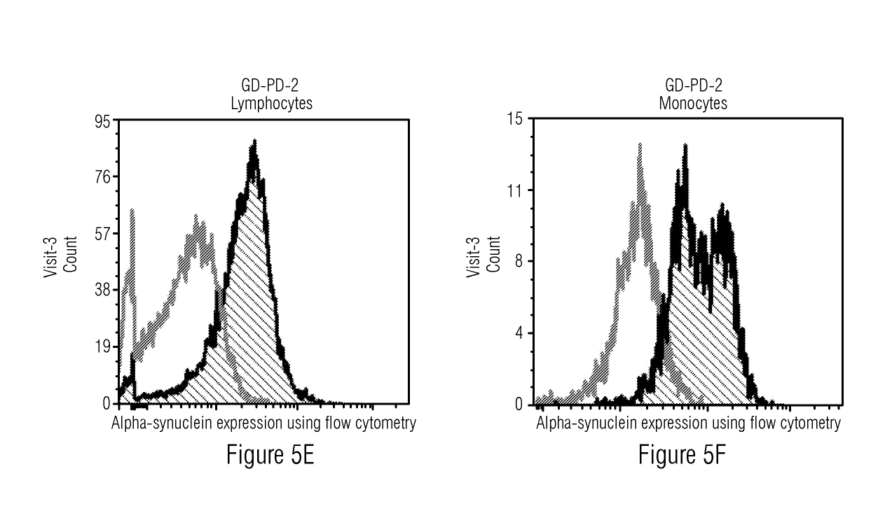

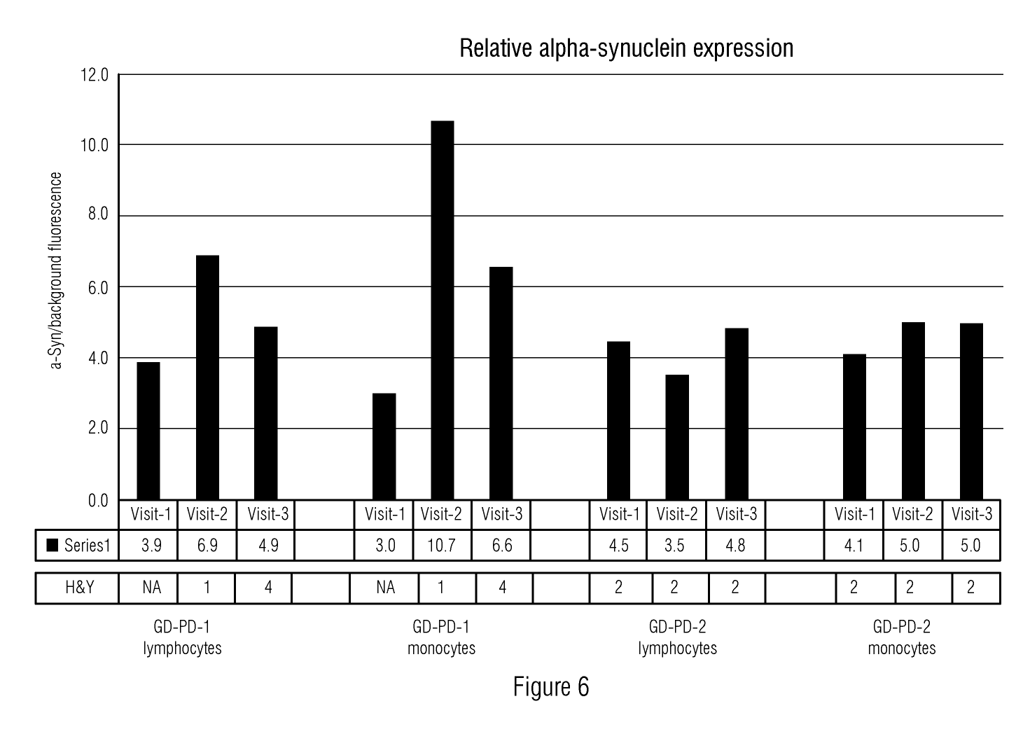

We then looked at the Alpha-Synuclein expression in two GD-PD subjects with differing disease progression to see if the expression of Alpha-Synuclein can reflect the clinical changes over a period of time (FIGS. 4&5). The relative Alpha-Synuclein expression over time for GD-PD1&2 is quantified in FIG. 6. In GD-PD-1, where the subject had rapid progression of PD symptoms, we noticed an appearance of a second peak in the monocytes. This peak increases with time in visit-3. This indicated that number of cells with accumulation of Alpha-Synuclein increased with time in GD-PD-1 subject. This result was not seen as aggressively in GD-PD-2 whose PD symptoms did not progress as quickly as in GD-PD-1 (FIG. 5).

### Significance and Claims:

Our results indicate, for the first time, that Alpha-Synuclein has a differential expression in cells of peripheral blood in subjects with GBA mutations and Parkinson's disease. This can be visualized by indirect immunofluorescence followed by flow cytometry which resulted in distinct pattern in subjects with PD and GBA associated PD. The assay utilizes easily accessible tissue type i.e., peripheral blood, minimally invasive method of sample collection and requires very little amount (1-5 ml) of peripheral blood. Therefore, we propose that this assay may be used to detect biomarkers (Alpha-Synuclein and Parkin) for diagnosis and disease progression for Parkinson disease in subjects. In one embodiment, these subjects has GBA gene mutations as well as other idiopathic PD and other synucleinopathies.

While specific embodiments are disclosed where the correlation between cell surface marker(s) or protein expression(s) and disease or disease risk is particularly useful, the assay may be applicable for diagnosis and risk assessment in patients without GBA gene mutations or previous disease state. Also, the methods of this disclosure are not limited to humans but is applicable to all mammals and animals such as commercially valuable livestock or domestic pets and also primates and other animals useful as an animal model of synucleinopathies (e.g., Parkinson Disease). The methods of the disclosure may be combined with other diagnostic and testing methods to achieve a synergistic and more reliable assessment of disease and/or disease risk.

### REFERENCES

Goker-Alpan et. al., Parkinsonism among Gaucher disease carriers. J Med Genet 2004: 41:937-940.

E Sidransky et. al., Multicenter analysis of glucocerebrosidase mutations in Parkinson's disease. N Engl J Med 2009: 361:1651-1661.

Mazzulli et. al., Gaucher disease glucocerebrosidase and a-synuclain form a bidirectional pathogenic loop in synucleinopathies. Cell 2011; 146: 37-52.

Giraldez-Perez et. al., Models of alpha-synuclein aggregation in Parkinson's disease. Acta Neuropathol Commun. 2014; 2: 176.

Swan and Saunders-Pullman, The association between beta-glucocerebrosidase mutations and parkinsonism. Curr Neurol Neurosci Rep., 2013; 13.

Kawahara et al., Alpha-Synuclein aggregates interfere with Parkin solubility and distribution. J Biol Chem 2008; 283:6979-6987.

Eblan M J, Nguyen J, Stubblefield B, et al. (2005) Glucocerebrosidase mutations in brain samples from subjects with parkinsonism. Mol Genet Metab. 84: 217-217.

Wolters E C, Francot C, Bergmans P, et al. (2000) Preclinical(premotor) Parkinson's disease. J Neurol. 247(Suppl 2): 103-109.

Guttman M, Burkholder J, Kish S J, Hussey D, et al. (1997) 11C RTI-32PET studies of the dopamine trasporter in early dopa-naïve Parkinson's disease: implications for the symptomatic threshold. Neurology 48: 1578-1583.

Morrish P K, Rakshi J S, Bailey D L, Sawle G V, Brooks D J. (1998) Measuring the rate of progression and estimating the preclinical periods of Parkinson's disease with [18 F] dopa PET. J Neurol Neurosurg and Psychiatry. 64:314-319.

All patents, patent applications, and references cited in this disclosure are incorporated by reference into this disclosure.

### EXAMPLES

### Example 1

### Subjects

Study included four cohorts: 1) Patients and carriers of Gaucher disease with confirmed disease causing mutations in GBA gene who have developed Parkinson disease symptoms (GD-PD), 2) Patients and carriers of Gaucher disease with no known Parkinson disease symptoms (GD-nonPD), 3) Patients with diagnosed Parkinson disease and no known GBA mutation or GD symptoms (nonGD-PD) and 4) Subjects with no known PD or GD symptoms (NonGD-nonPD).

### Example 2

### Isolation of Peripheral Blood Mononuclear Cells (PBMCs)

PBMCs are extracted from 3-5 ml peripheral blood using Ficoll-paque (GE health care). 2-4 ml of whole blood is diluted 1:2 using Phosphate buffered saline (PBS) containing 2% fetal bovine serum (FBS) and overlayed onto Ficoll solution in 15 ml leucosep tube. The tubes are centrifuged at 2000RCF for 10 minutes with no brakes. The layer containing PBMCs is transferred into a fresh 15 ml tube and washed with PBS+2% FBS. The cells are then resuspended in cell freezing medium (50% RPMI+40% FBS+10% DMSO) and stored at −150° C. in a freezer until their use.

### Example 3

### Immunostaining

The cryopreserved PBMCs were thawed at 37° C. for 2 minutes, washed and resuspended in PBS+2% FBS and used for immunostaining. Approximately 5×10⁵ cells per tube were fixed and permeabilized using Fix & Perm Cell Fixation and Cell Permeabilization kit (Thermo Fisher Scientific) as per manufacturer's instructions. Rabbit anti-human-Alpha-Synuclein and Rabbit-anti-human-Parkin antibodies (catalog numbers 701085 and PA5-13399 respectively, from Thermo Fisher Scientific) were added to individual tubes for intracellular staining for 20 minutes at room temperature. No primary antibody was added to the control tubes. The cells were then washed with 2 ml of PBS+2% FBS. The tubes were centrifuged to remove wash buffer. The cells were resuspended in PBS+2% FBS containing Goat anti-Rabbit antibody conjugated with Alexa fluor 647 which acted as secondary antibody and incubated at room temperature for 20 minutes. The cells were then washed in PBS+2% FBS and acquired on Flow cytometer (BD accuri). The results were analyzed using FCS Express 6 software (Denovo software). Using FSC vs SSC scatter plot, lymphocytes and monocytes were gated and were analyzed for subsequent Alpha-Synuclein expression or Parkin expression using scatter plots and histogram overlays.

Although the disclosure has been described with reference to various example embodiments, it should be understood that various modifications can be made without departing from the spirit of the disclosure. Accordingly, the scope of the disclosure should be determined with reference to the appended claims, along with the full scope of equivalents to which such claims are entitled. The disclosures of all cited articles and references, including patent applications and publications, are incorporated herein by reference for all purposes.

We claim:

1. A method to diagnose synucleinopathy in a first subject comprising the steps of:
a) detecting a lymphocyte expression level of Alpha-Synuclein or a monocyte expression level of Alpha-Synuclein in a first subject;
b) comparing the lymphocyte expression level of Alpha-Synuclein or the monocyte expression level of Alpha-Synuclein with a second subject who does not have synucleinopathy wherein an increase in the expression level of Alpha-Synuclein in the first subject relative to the second subject indicates that the subject has synucleinopathy.

2. The method of claim 1 wherein said synucleinopathy is at least one selected from the group consisting of Parkinson Disease, Parkinsonism, dementia with Lewy bodies; and multiple system atrophy.

3. The method of claim 1 wherein said detecting step is detecting from a blood sample or a peripheral blood sample of said subject.

4. The method of claim 1 wherein said detecting step is detecting from a peripheral blood mononuclear cells sample collected from said first subject.

5. The method of claim 1 wherein a ratio of the expression level of Alpha-Synuclein in the first subject to the expression level of Alpha-Synuclein in the second subject is at least 1.5 to 1 for lymphocyte cells or at least 2 to 1 for lymphocyte cells.

6. The method of claim 1 wherein a ratio of the expression level of Alpha-Synuclein in the first subject to the expression level of Alpha-Synuclein in the second subject is at least 1.5 to 1 for monocyte cells or at least 2 to 1 for monocyte cells.

7. The method of claim 1, wherein the step of detecting is performed by flow cytometry.

8. The method of claim 1, wherein the step of detecting is performed with an anti Alpha-Synuclein antibody.

9. The method of claim 1 wherein said first subject or said second subject has Gaucher Disease or has Gaucher carrier status.

10. The method of claim 9 wherein said subject has at least one mutated GBA gene.

11. The method of claim 10 wherein the at least one mutated GBA gene encodes a glucocerebrosidase with a reduced glucosylceramide cleavage activity.

12. A method for monitoring the progression of synucleinopathy or synucleinopathy status in a subject comprising the steps of:
a) detecting a first lymphocyte Alpha-Synuclein expression level or a first monocyte Alpha-Synuclein expression level from a first sample from said subject;
b) determining the progression of synucleinopathy in the subject based on comparing said first lymphocyte Alpha-Synuclein expression level or said first monocyte Alpha-Synuclein expression level with a second expression level from a second sample from the same subject collected earlier or from a second subject with synucleinopathy; wherein a high first expression level relative to said second expression level indicates a more rapid progression of synucleinopathy and a low expression level relative to said second expression level indicates a less rapid progression of synucleinopathy.

13. The method of claim 12 wherein said synucleinopathy is at least one selected from the group consisting of Parkinson Disease, Parkinsonism, dementia with Lewy bodies; and multiple system atrophy.

14. The method of claim 12, wherein said detecting step is detecting from a blood sample or a peripheral blood sample of said subject.

15. The method of claim 12, wherein said detecting step comprises analysis by flow cytometry.

16. The method of Clam 12, wherein the step of detecting is performed with an anti-Alpha-Synuclein antibody.

17. The method of claim 12, wherein said subject has Gaucher Disease or has Gaucher carrier status.

18. The method of claim 17, wherein said subject has at least one mutated GBA gene.

19. The method of claim 18, wherein the at least one mutated GBA gene encodes a glucocerebrosidase with a reduced glucosylceramide cleavage activity.

20. A kit for the diagnosis of synucleinopathy in a subject comprising:
a) a first reagent for the detection of Alpha-Synuclein expression a cell; and
b) a second reagent selected from the group consisting of a positive control reagent and a negative control reagent.

21. The method of claim 20 wherein said synucleinopathy is at least one selected from the group consisting of Parkinson Disease, Parkinsonism, dementia with Lewy bodies; and multiple system atrophy.

22. The kit of claim 20, wherein the positive control reagent comprises lymphocytes or monocytes from peripheral blood of a second subject which has at least one selected from the group consisting of synucleinopathy, Gaucher Disease and Gaucher carrier status.

23. The kit of claim 20, wherein the negative control reagent comprises lymphocytes and monocytes from peripheral blood of a third subject which does not have Gaucher Disease, Gaucher carrier status or synucleinopathy.
